(19) United States
(12) Patent Application Publication
(10) Pub. No.: US 2022/0291243 A1
(43) Pub. Date: Sep. 15, 2022

(54) ANALYZING HORMONE PANELS FOR THE PREDICTION OF SPONTANEOUS PRETERM DELIVERY

(71) Applicant: Garbha Health, Inc., Phoenix, AZ (US)

(72) Inventor: Avinash Patil, Phoenix, AZ (US)

(21) Appl. No.: 16/765,523

(22) PCT Filed: Dec. 4, 2019
(86) PCT No.: PCT/US2019/064508

§ 371 Date: May 20, 2020

(51) Int. Cl. G01N 33/74; G01N 33/68; G16H 10/40

(52) U.S. Cl. CPC G01N 33/743; G01N 33/689; G16H 10/40; G01N 2800/368; G01N 2800/60

(57) ABSTRACT

Disclosed are methods for analyzing hormone profiles in a pregnant female to determine the pregnant females susceptibilty to spontaneous preterm delivery. In particular, disclosed are methods for identifying a pregnant female who is susceptible to spontaneous preterm delivery based on a formula including ratios of steroids in samples obtained from the pregnant female. Further, the methods can include treating the pregnant female identified susceptible to spontaneous preterm delivery.

### CROSS REFERENCE TO RELATED APPLICATIONS

This application is a US National Phase Application of PCT/US2019/064508 filed Dec. 4, 2019, which is hereby incorporated by reference in its entirety.

### BACKGROUND OF THE DISCLOSURE

The present disclosure relates generally to analyzing hormone panels, and in particular, steroid panels, for assessing the risk of preterm delivery in a pregnant female. More particularly, the present disclosure relates to assessing risk of spontaneous preterm delivery in a pregnant female using ratios of endogenous steroids in samples obtained from a pregnant female.

Preterm birth is a major public health problem, leading to lifelong morbidities in premature newborns and high expenditures for health care systems and insurance companies. Each year, an estimated 15 million babies are born preterm (before 37 weeks gestation) according to the World Health Organization. Globally, preterm birth is the leading cause of newborn deaths (babies in the first four weeks of life) and the second leading cause of death in children under five years. Nationally, preterm delivery prior to 32 weeks gestation is associated with an 80-fold increase in infant mortality compared to women delivery between 39-41 weeks. Complications arising from preterm birth include acute respiratory, gastrointestinal, immunologic, central nervous system, hearing, and vision problems, as well as longer-term motor, cognitive, visual, hearing, behavioral, social-emotional, health, and growth problems. Many survivors face a lifetime of disability, including learning disabilities and visual and hearing problems.

If a pregnant woman is determined to be at risk for preterm birth, health care providers can implement various clinical strategies that may include surgical procedures such as cervical cerclage and cervical pessaries, preventive medications, restrictions on sexual activity and/or other physical activities, and alterations of treatments for chronic conditions that increase the risk of preterm labor.

Women identified as high-risk can be scheduled for more intensive surveillance and interventions. Very few technologies exist to identify women at risk for preterm birth who could benefit from additional interventions. The tools that are currently available have limited sensitivity/specificity or identify molecular changes associated with preterm labor without offering any interventions to mitigate this process. Current strategies for risk assessment are based on the obstetric and medical history and clinical examination, but these strategies are only able to identify a small percentage of women who are at risk for preterm delivery. Clinically available tools for risk assessment are available in the mid-second trimester, after the period of maximal benefit from interventions. Reliable early identification of risk for preterm birth would allow for appropriate monitoring and clinical management to prevent preterm delivery.

Further, only one medication has been approved by the FDA for the prevention of recurrent spontaneous preterm delivery: 17α-hydroxyprogesterone caproate (HPC). A landmark prospective trial of HPC in women with a prior history of spontaneous preterm birth (sPTB) demonstrated a 30-40% reduction in the risk of recurrent sPTB. However, 40% of preterm deliveries occur in first-time pregnancies and 70% of pregnant women delivering prematurely in their second pregnancy had full-term deliveries in their first pregnancy. Further, HPC is not uniformly effective in all patients, and identification of biomarkers to predict therapeutic response is still in progress.

Accordingly, there exists a need for alternative tools for assessing risk factors in the occurrence of spontaneous preterm delivery.

### BRIEF DESCRIPTION OF THE DISCLOSURE

The present disclosure is generally directed to methods for analyzing a hormonal panel in a pregnant female to assess whether a pregnant female is susceptible to spontaneous preterm delivery. More particularly, in one embodiment, the present disclosure is directed to methods for assessing panels in a pregnant female to determine whether the female is susceptible to spontaneous preterm delivery based on the formula of:

$$Score = \frac{[ 11 - deoxycorticosterone ]}{{( \frac{{\sum }_{i = 1}^{j} {{log}_{10} [ steroid ]}_{i}}{j} )}^{{\prod }_{i = 1}^{p} {{log}_{10} [ n e u r o steroid ]}_{i}}}$$

wherein j is the number of steroid molecules measured; p represents the number of neurosteroid molecules measured; and i represents the individual steroid or neurosteroid molecules up to a total of j or p, respectively.

In suitable embodiments, the methods use a single steroid and a single neurosteroid in the denominator of the formula. For example, the methods for assessing panels in a pregnant female to determine whether the female is susceptible to spontaneous preterm delivery is based on the formula of:

$$Score = \frac{[ 11 - deoxycorticosterone ]}{{( {log}_{10} [ steroid ] )}^{{log}_{10} [ n e u r o steroid ]}} .$$

In one particularly suitable embodiment, the present disclosure uses 16-hydroxyprogesterone as the steroid in the denominator of the formula. In another particularly suitable embodiment, the present disclosure uses 11-deoxycoritsol as the steroid in the denominator of the formula, to assess the risk of a pregnant female as being susceptible to spontaneous preterm delivery. It has been found that the methods of the present disclosure are effective at identifying susceptibility of spontaneous preterm delivery in both women with a history of preterm delivery and in women without a history of preterm delivery.

Additionally, the present disclosure is directed to treating the pregnant female once identified as being susceptible to spontaneous preterm delivery. Particularly, the present disclosure is directed to administering an effective amount of 17α-hydroxyprogesterone caproate (HPC), monohydroxylated HPC (HPC-OH), atosiban, nifedipine, terbutaline, eplerenone, fluoxetine and norfluoxetine, other mineralocorticoid specific antagonists, modulators of cytochrome P450 function (e.g., CYP2D6, CYP2C9, CYP3A4), and combinations thereof to a pregnant female identified as susceptible to spontaneous preterm delivery.

In one aspect, the present disclosure is directed to a method for analyzing a hormone panel in a pregnant female, the method comprising: obtaining a sample from the pregnant female; detecting a concentration of 11-deoxycorticosterone in the sample; detecting a concentration of a neurosteriod in the sample; detecting a concentration of a steroid in the sample; and calculating a score for the hormone profile using the formula:

$$Score = \frac{[ 11 - deoxycorticosterone ]}{{( {log}_{10} [ steroid ] )}^{{log}_{10} [ n e u r o steroid ]}} .$$

In some embodiments, the steroid is selected from 16α-hydroxyprogesterone, corticosterone, 18-hydroxycortiosterone, aldosterone, 11-deoxycortisol, estradiol, testosterone, and 17-hydroxyprogesterone, cortisol, and combinations thereof. In some embodiments, the neurosteriod is selected from isopregnanolone, epipregnanolone, pregnenolone sulfate, pregnanolone, allopregnanolone, tetrahydrodeoxycorticosterone, and combinations thereof.

In another aspect, the present disclosure is directed to a method for treating a pregnant female susceptible to spontaneous preterm delivery. The method comprises: using the above formula to analyze a hormone panel and calculate a score. If the score of the calculated formula is less than 1.1, then administering an effective amount of a compound selected from the group consisting of 17α-hydroxyprogesterone caproate (HPC), monohydroxylated HPC (HPC-OH), atosiban, nifedipine, terbutaline, eplerenone, fluoxetine and norfluoxetine, other mineralocorticoid specific antagonists, modulators of cytochrome P450 function (e.g., CYP2D6, CYP2C9, CYP3A4), and derivatives thereof, and combinations thereof.

In accordance with some embodiments of the present disclosure, methods have been discovered that surprisingly allow for identifying and treating a pregnant female who is susceptible to spontaneous preterm delivery. Significantly, the methods of the present disclosure allow for determining whether a pregnant female is susceptible to having a spontaneous preterm delivery based on samples obtained weeks, and even months, prior to delivery. The methods of the present disclosure significantly allow for the identification of a pregnant female as being susceptible for spontaneous preterm birth allowing for appropriate monitoring and clinical management to prevent spontaneous preterm delivery.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a ROC graph depicting various ratios of 11-deoxycorticosterone (cortexone) and/or 16α-Hydroxyprogesterone (16α-OHP) and isopregnanolone and spontaneous preterm birth (sPTB) at less than 32 weeks in Epoch 1. AUC 0.830, 95% CI 0.697-0.963 (p=0.002), 100% sensitivity, 54% specificity (threshold≤1.09).

FIG. 2 is a ROC graph depicting various ratios of 11-deoxycorticosterone (cortexone) and/or estradiol and isopregnanolone and spontaneous preterm birth (sPTB) at less than 32 weeks in Epoch 1. AUC 0.863, 95% CI 0.748-0.979 (p=0.001), 100% sensitivity, 57% specificity (threshold≤0.25).

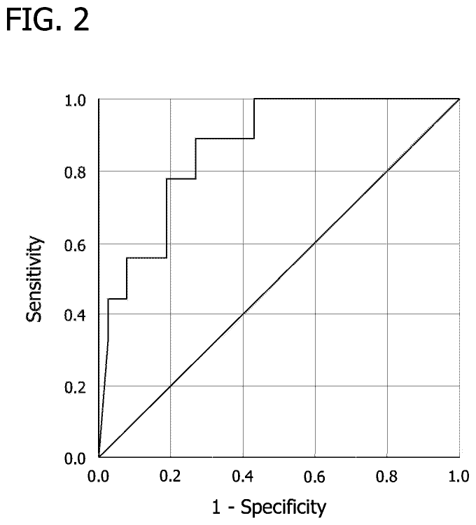

FIG. 3 is a ROC graph depicting various ratios of 11-deoxycorticosterone (cortexone) and/or testosterone and isopregnanolone and spontaneous preterm birth (sPTB) at less than 32 weeks in Epoch 1. AUC 0.794, 95% CI 0.638-0.951 (p=0.007), 100% sensitivity, 38% specificity (threshold≤1.19).

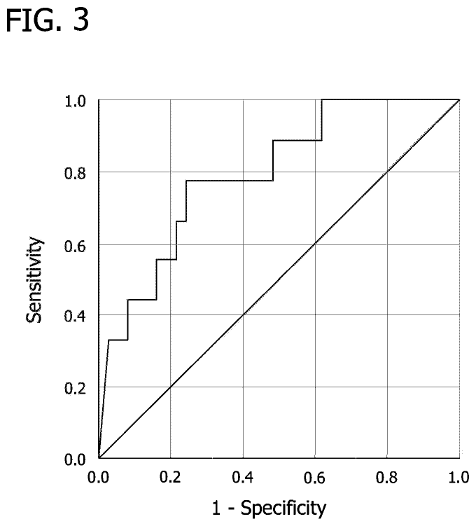

FIG. 4 is a ROC graph depicting various ratios of 11-deoxycorticosterone (cortexone) and/or cortisol and isopregnanolone and spontaneous preterm birth (sPTB) at less than 32 weeks in Epoch 1. AUC 0.809, 95% CI 0.665-0.953 (p=0.004), 100% sensitivity, 51% specificity (threshold≤0.065).

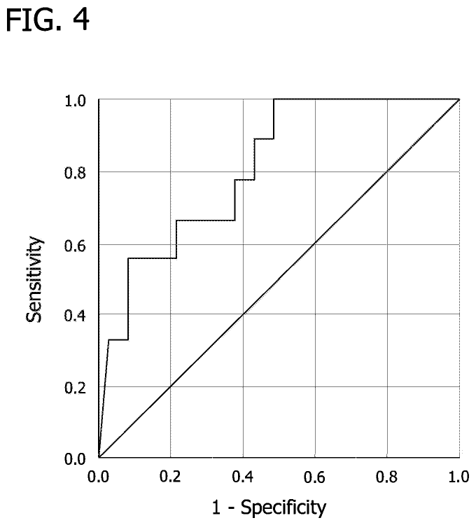

FIG. 5 is a ROC graph depicting various ratios of 11-deoxycorticosterone (cortexone) and/or progesterone and isopregnanolone and spontaneous preterm birth (sPTB) at less than 32 weeks in Epoch 1. AUC 0.798, 95% CI 0.635-0.961 (p=0.007), 100% sensitivity, 30% specificity (threshold≤1.98).

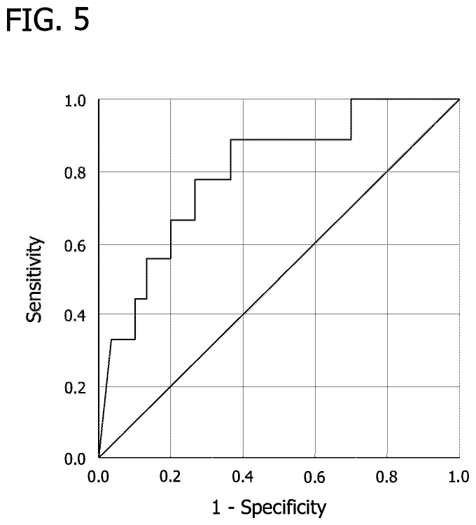

FIG. 6 is a ROC graph depicting various ratios of 11-deoxycorticosterone (cortexone) and/or corticosterone and isopregnanolone and spontaneous preterm birth (sPTB) at less than 32 weeks in Epoch 1. AUC 0.787, 95% CI 0.615-0.959 (p=0.013), 100% sensitivity, 34% specificity (threshold≤0.69).

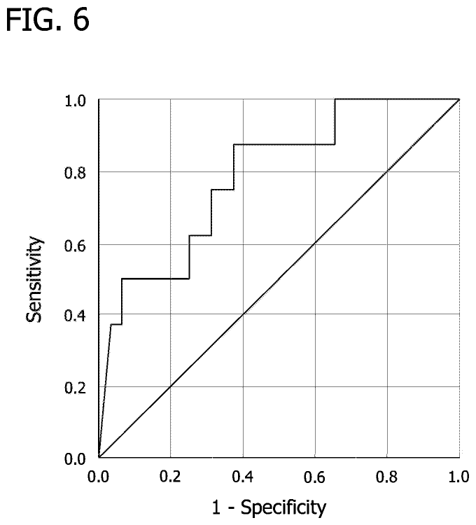

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the suitable methods and materials are described below.

As used in this application, including the appended claims, the singular forms “a,” “an,” and “the” include plural references, unless the content clearly dictates otherwise, and are used interchangeably with “at least one” and “one or more.”

As used herein, “spontaneous preterm delivery” and “spontaneous preterm birth” are used interchangeably herein to refer to delivery or birth at a gestational age less than 37 completed weeks. Other commonly used subcategories of spontaneous preterm birth delineate moderately preterm (birth at 33 to 37 weeks of gestation), very preterm (birth at less than 33 weeks of gestation), and extremely preterm (birth at less than 28 weeks of gestation, for example, from 24 weeks to 28 weeks gestation or even earlier).

A number of methods can be used to determine the amount of a hormone, including mass spectrometry approaches, such as MS/MS, LC-MS/MS, multiple reaction monitoring (MRM) or SRM and product-ion monitoring (PIM) and also including antibody based methods such as immunoassays such as Western blots, enzyme-linked immunosorbant assay (ELISA), immunopercipitation, immunohistochemistry, immunofluorescence, radioimmunoassay, dot blotting, and FACS.

A detectable label can be used in the assays described herein for direct or indirect detection of the hormones in the methods of the present disclosure. A wide variety of detectable labels can be used, with the choice of label depending on the sensitivity required, ease of conjugation with the antibody, stability requirements, and available instrumentation and disposal provisions. Those skilled in the art are familiar with selection of a suitable detectable label based on the assay detection of the hormones in the methods of the present disclosure. Suitable detectable labels include, but are not limited to, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), enzymes (e.g., luciferase, horseradish peroxidase, alkaline phosphatase, etc.), nanoparticles, biotin, digoxigenin, metals, and the like.

The predictive ability of a model can be evaluated according to its ability to provide a quality metric, e.g. AUROC (area under the ROC curve) or accuracy, of a particular value, or range of values. Area under the curve measures are useful for comparing the accuracy of a classifier across the complete data range.

Classifiers with a greater AUC have a greater capacity to classify unknowns correctly between two groups of interest. In some embodiments, a desired quality threshold is a predictive model that will classify a sample with an accuracy of at least 0.5, at least 0.55, at least 0.6, at least 0.7, at least 0.75, at least 0.8, at least 0.85, at least 0.9, at least 0.95, or higher. As an alternative measure, a desired quality threshold can refer to a predictive model that will classify a sample with an AUC of at least 0.7, at least 0.75, at least 0.8, at least 0.85, at least 0.9, or higher.

The term “measurement” suitably comprises a qualitative, semi-qualitative or a quantitative measurement of hormones selected from progesterone, 16α-hydroxyprogesterone, 6β-hydroxyprogesterone, 6α-hydroxyprogesterone, 17-hydroxyprogesterone, 11-deoxycortisol, cortisol, 11-deoxycorticosterone, 17-deoxycortisol, androstenedione, testosterone, estradiol, 20α-dihydroprogesterone, 17α,20α-dihydroxyprogesterone, and isopregnanolone in a sample. In a suitable embodiment the measurement is a semi-quantitative measurement, i.e., score is determined by the ratios of steroids selected from progesterone, 16α-hydroxyprogesterone, 6β-hydroxyprogesterone, 6α-hydroxyprogesterone, 17-hydroxyprogesterone, 11-deoxycortisol (cortexolone), cortisol, 11-deoxycorticosterone (cortexone), 17-deoxycortisol, androstenedione, testosterone, estradiol, 20α-dihydroprogesterone, 17α,20α-dihydroxyprogesterone, isopregnanolone, pregnanolone, allopregnanolone, tetrahydrodeoxycorticosterone, epipregnanolone, pregnenolone sulfate, and the like, and combinations thereof to be above or below a cut-off (threshold) value. As the skilled artisan will appreciate, in a Yes-(presence) or No-(absence) assay, the assay sensitivity is usually set to match the cut-off value. A cut-off value can, for example, be determined from the testing of a group of healthy individuals. Suitably, the cut-off is set to result in a specificity of 90%, also suitable, the cut-off is set to result in a specificity of 95%, or also suitable, the cut-off is set to result in a specificity of 98%. A value below the cut-off value can, for example be indicative for spontaneous preterm delivery. In particular, a value below the cut-off value can, for example, be indicative for spontaneous preterm delivery at less than 37 weeks gestation. In particular, a value below the cut-off value can, for example, be indicative for spontaneous preterm delivery at less than 28 weeks gestation. In particular, a value below the cut-off value can, for example, be indicative for spontaneous preterm delivery at less than 26 weeks gestation, including less than 24 weeks, including from about 24 weeks to about 28 weeks. In particular, a value below the cut-off value can, for example, be indicative for spontaneous preterm delivery at less than 24 weeks gestation, including less than 20 weeks, including from about 10 weeks to about 24 weeks, including from about 10 weeks to about 16 weeks, and including from about 10 weeks to about 12 weeks. Alternatively, a value above the cut-off value can, for example, be indicative for less susceptibility to spontaneous preterm delivery. In particular, a value above the cut-off value can, for example, be indicative for less susceptibility to spontaneous preterm delivery at less than 28 weeks gestation. In particular, a value above the cut-off value can for example be indicative for less susceptibility to spontaneous preterm delivery at less than 26 weeks gestation. In particular, a value above the cut-off value can for example be indicative for less susceptibility to spontaneous preterm delivery at less than 24 weeks gestation. In particular, a value above the cut-off value can for example be indicative for less susceptibility to spontaneous preterm delivery at less than 20 weeks gestation, including from about 10 weeks to about 24 weeks, including from about 10 weeks to about 16 weeks, and including from about 10 weeks to about 12 weeks.

In another suitable embodiment, the cut-off is set to result in a sensitivity of 90%, also suitable, the cut-off is set to result in a sensitivity of 95%, or also suitable, the cut-off is set to result in a sensitivity of 98%.

It has surprisingly been determined that the formula of:

$$Score = \frac{[ 11 - deoxycorticosterone ]}{{( {log}_{10} [ steriod ] )}^{l o {g}_{10} [ n e u r o s t e riod ]}} .$$

can indicate a risk of spontaneous preterm delivery in a subject. Statistical models permit ROC curve analysis of the multi marker assay, and the results confirm the diagnostic accuracy. Significantly, none of the hormones when analyzed alone indicates spontaneous preterm delivery.

In the formula, the steroid for use in the denominator of the formula includes one or more of 16α-hydroxyprogesterone, corticosterone, 18-hydroxycortiosterone, aldosterone, 11-deoxycortisol, estradiol, testosterone, and 17-hydroxyprogesterone, cortisol. In particular, the steroid can be one or more of 16α-hydroxyprogesterone, 11-deoxycortisol, estradiol, testosterone, and 17-hydroxyprogesterone.

The neurosteriod is selected from isopregnanolone, epipregnanolone, pregnenolone sulfate, pregnanolone, allopregnanolone, tetrahydrodeoxycorticosterone, and combinations thereof.

Well-known mathematical methods for analyzing the ratios of combinations of 11-deoxycorticosterone, 16α-hydroxyprogesterone, corticosterone, 18-hydroxycortiosterone, aldosterone, 11-deoxycortisol, estradiol, testosterone, and 17-hydroxyprogesterone, cortisol, and isopregnanolone, employ methods like, discriminant analysis (DA) (i.e., linear-, quadratic-, regularized-DA), Kernel Methods (i.e., SVM), Nonparametric Methods (i.e., k-Nearest-Neighbor Classifiers), PLS (Partial Least Squares), Tree-Based Methods (i.e., Logic Regression, CART, Random Forest Methods, Boosting/Bagging Methods), Generalized Linear Models (i.e., Logistic Regression), Principal Components based Methods (i.e., SIMCA), Generalized Additive Models, Fuzzy Logic based Methods, Neural Networks and Genetic Algorithms based Methods.

Accuracy of a risk assessment method is best described by its receiver-operating characteristics (ROC). The ROC graph is a plot of all of the sensitivity/specificity pairs resulting from continuously varying the decision thresh-hold over the entire range of data observed. Diagnostic accuracy measures the test's ability to correctly distinguish two different conditions of the subjects being investigated such as, for example, health and disease. The ROC plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1-specificity [defined as (number of false-positive results)/(number of true-negative+number of false-positive results)]. It is an index of specificity and is calculated entirely from the unaffected subgroup. Because the true- and false-positive fractions are calculated entirely separately using the test results from two different subgroups, the ROC plot is independent of the prevalence of disease in the sample.

Each point on the ROC plot represents a sensitivity/1-specificity pair corresponding to a particular decision threshold. A test with perfect discrimination (no overlap in the two distributions of results) has a ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is remedied by reversing the criterion for “positivity” from “greater than” to “less than” or vice versa.) Qualitatively, the closer the plot is to the upper left corner, the higher the overall accuracy of the test. One particularly suitable way to quantify the diagnostic accuracy of a laboratory test is to express its performance by a single number. Such an overall parameter, e.g., is the so-called “total error” or alternatively the “area under the curve=AUC”. The most common global measure is the area under the ROC plot. By convention, this area is always ≥0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

In accordance with the present disclosure, methods for analyzing a hormone panel in pregnant female subjects are disclosed. The methods include: obtaining a sample from the pregnant female; detecting a concentration of 11-deoxycorticosterone in the sample; detecting a concentration of at least one neurosteroid in the sample; detecting a concentration of a steroid in the sample; and calculating a score for the hormone profile. In particular, a score is calculated during the first, second and/or third trimesters using the formula:

$$Score = \frac{[ 11 - deoxycorticosterone ]}{{( {log}_{10} [ steriod ] )}^{l o {g}_{10} [ n e u r o s t e riod ]}} .$$

In particular, a score is calculated during the first, second and/or third trimesters using the formula:

$$Score = \frac{[ 11 - deoxycorticosterone ]}{{( {log}_{10} [ steriod ] )}^{l o {g}_{10} [ isopregnanolone ]}} .$$

By way of example, the present disclosure is directed to a method for analyzing a hormone panel in a pregnant female during the first or second trimester, the method including: obtaining a sample from the pregnant female; detecting a concentration of 11-deoxycorticosterone in the sample; detecting a concentration of a neurosteriod in the sample; detecting a concentration of a steroid selected from the group consisting of 16α-hydroxyprogesterone, corticosterone, 18-hydroxycortiosterone, aldosterone, 11-deoxycortisol, estradiol, testosterone, and 17-hydroxyprogesterone, cortisol, and combinations thereof in the sample; and calculating a score for the hormone profile. The score being calculated using the formula:

$$Score = \frac{[ 11 - deoxycorticosterone ]}{{( {log}_{10} [ 16 α - hydroxyprogesterone ] )}^{l o {g}_{10} [ isopre g n anolone ]}} .$$

In another example, the present disclosure is directed to a method for analyzing a hormone panel in a pregnant female during the second trimester, the method including: obtaining a sample from the pregnant female; detecting a concentration of 11-deoxycorticosterone in the sample; detecting a concentration of a neurosteriod in the sample; detecting a concentration of a steroid in the sample; and calculating a score for the hormone profile. The score being calculated using the formula:

$$Score = \frac{[ 11 - deoxycorticosterone ]}{{( {log}_{10} [ estradiol ] )}^{l o {g}_{10} [ isopre g n anolone ]}} .$$

The methods of the present disclosure allow for identifying a pregnant female as being susceptible to spontaneous preterm delivery based on samples obtained weeks, and even months, prior to delivery. The methods can be utilized with pregnant females that are in their first pregnancy, have had preterm delivery in one or more prior pregnancies or have had full-term deliveries in one or more prior pregnancies. The pregnant female is identified as being susceptible to spontaneous preterm delivery if the calculated score from one or more of the above formulas is less than 1.5, including less than 1.1. More particularly, in one embodiment, when the formula is

$$Score = \frac{[ 11 - deoxycorticosterone ]}{{( {log}_{10} [ 16 α - hydroxyprogesterone ] )}^{l o {g}_{10} [ isopre g n anolone ]}} ,$$

the pregnant female is identified as being susceptible to spontaneous preterm delivery if the calculated score is less than 1.5, including less than 1.1. In another embodiment, when the formula is

$$Score = \frac{[ 11 - deoxycorticosterone ]}{{( {log}_{10} [ 11 - deoxycortisol ] )}^{l o {g}_{10} [ isopre g n anolone ]}}$$

the pregnant female is identified as being susceptible to spontaneous preterm delivery if the calculated score is less than 1.5, including less than 1.1.

In some embodiments, the formula used in the methods can further include at least one additional steroid and/or neurosteroid and is the formula:

$$Score = \frac{[ 11 - deoxycorticosterone ]}{{( \frac{{\sum }_{i = 1}^{j} {{log}_{10} [ steroid ]}_{i}}{j} )}^{{\prod }_{i = 1}^{p} {{log}_{10} [ neurosteroid ]}_{i}}}$$

wherein j is the number of steroid molecules measured; p represents the number of neurosteroid molecules measured; and i represents the individual steroid or neurosteroid molecules up to a total of j or p, respectively.

As used herein, “at least one additional steroid” refers to a second steroid, a third steroid, a fourth steroid, and so-forth, including combinations of the second steroid, the third steroid, the fourth steroid, and so-forth. Thus, in one embodiment, the score can be calculated using a first additional steroid, for example. In another embodiment, the score can be calculated using a first additional steroid and a second additional steroid, for example. In another embodiment, the score can be calculated using a first additional steroid, a second additional steroid, a third additional steroid, and so-forth.

Particularly suitable steroids include 16α-hydroxyprogesterone, corticosterone, 18-hydroxycortiosterone, aldosterone, 11-deoxycortisol, estradiol, testosterone, and 17-hydroxyprogesterone, cortisol, 11-deoxycorticosterone, and isopregnanolone.

Similarly, more than one neurosteroid can be used in above formula for use in the methods. Accordingly, in some embodiments, the score can be calculated using one neurosteroid, for example In another embodiment, the score can be calculated using a first neurosteroid and a second neurosteroid, for example In another embodiment, the score can be calculated using a first neurosteroid, a second neurosteroid, a third neurosteroid, and so-forth. Suitable neurosteriods include isopregnanolone, epipregnanolone, pregnenolone sulfate, pregnanolone, allopregnanolone, tetrahydrodeoxycorticosterone, and combinations thereof.

The method can further include determining a change in a concentration of at least one additional biomarker selected from insulin-like growth factor binding protein 4, sex-hormone binding globulin (SHBG), lipopolysaccharide-binding protein (LBP), lipopolysaccharide-binding protein (LBP) precursor, prothrombin (THRB), complement component C5 (C5 or CO5), plasminogen (PLMN), complement component C8 gamma chain (C8G or CO8G), Complement factor B, Ectonucleotide pyrophosphatase/phosphodiesterase family member 2, Gelsolin, N-acetylmuramoyl-L-alanine amidase, N-acetylmuramoyl-L-alanine amidase precursor, Hyaluronan-binding protein 2, BPI fold-containing family B member 1, complement component C8 alpha chain, apolipoprotein A-II, Ectonucleotide pyrophosphatase/phosphodiesterase family member 2, profiling-1, pro-neuropeptide Y, complement component C8 beta chain, coagulation factor XIIII B chain, N-acetylmuramoyl-L-alanine amidase, inter-alpha-trypsin inhibitor heavy chain H4, inter-alpha-trypsin inhibitor heavy chain H3 preproprotein, leucyl-cystinyl aminopeptidase, alpha-2-HS-glycoprotein, 5′-AMP-activated protein kinase subunit gamma-3, afamin precursor, alpha-1-antichymotrypsin precursor, alpha-1B-glycoprotein precursor, alpha-2-antiplasmin isoform a precursor, alpha-2-HS-glycoprotein preproprotein, alpha-2-HS-macroglobulin precursor, angiotensinogen preproprotein, antithrombin-III precursor, apolipoprotein A-II preproprotein, apolipoprotein A-IV precursor, apolipoprotein B-100 precursor, apolipoprotein C-I precursor, apolipoprotein C-II precursor, apolipoprotein C-III precursor, apolipoprotein E precursor, ATP-binding, cassette sub-family D member 4, ATP-binding cassette sub-family F member 3, beta-2-glycoprotein 1 precursor, beta-Ala-His dipeptidase precursor, biotiinidase precursor, carboxypeptidase B2 preproprotein, carboxypeptidase N catalytic chain precursor, carboxypeptidase N subunit 2 precursor, catalase, ceruloplasmin precursor, cholinesterase precursor, clusterin preproprotein, coagulation factor IX preproprotein, coagulation factor VII isoform a, coagulation factor VII isoform a preproprotein, coagulation factor X preproprotein, coagulation factor XIII B chain, coiled-coil domain-containing protein 13, complement C1q subcomponent subunit A precursor, complement C1q subcomponent subunit B precursor, complement C1q subcomponent subunit C precursor, complement C1r subcomponent precursor, complement C1s subcomponent precursor, complement C2 isoform 3, complement C3 precursor, complement C4-A isoform 1, complement C5 preproprotein, component C6 precursor, component C7 precursor, component C8 alpha chain precursor, complement component C9 precursor, complement factor B preproprotein, complement factor H isoform a precursor, complement factor H isoform b precursor, complement factor H H-related protein 1 precursor, complement factor I preproprotein, conserved oligomeric Golgi complex subunit 6 isoform, corticosteroid-binding globulin precursor, C-reactive protein precursor, dopamine beta-hydroxylase precursor, double-stranded RNA-specific editase B2, dual oxidase 2 precursor, FERM domain-containing protein 8, fetuin-B precursor, ficolin-3 isoform 1 precursor, gastric intrinsic factor precursor, gelsolin isoform d, glutathione peroxidase 3 precursor, hemopexin precursor, heparin cofactor 2 precursor, hepatocyte cell adhesion molecule precursor, hepatocyte growth factor activator preproprotein, histidine-rich glycoprotein precursor, hyaluronan-binding protein 2 isoform 1 preproprotein, inactive caspase-12 insulin-degrading enzyme isoform 1, insulin-like growth factor-binding protein complex acid labile subunit isoform 2 precursor, inter-alpha-trypsin inhibitor heavy chain H1 isoform a precursor, inter-alpha-trypsin inhibitor heavy chain H2 precursor, Inter- alpha-trypsin inhibitor heavy chain H4 isoform 1 precursor, kallistatin precursor, kininogen-1 isoform 2 precursor, leucine-rich alpha-2-glycoprotein precursor, lumican precursor, m7(ipppX diphosphatase, matrix metalloproteinase-19 isoform 1 preproprotein, MET domain-containing protein 1, monocyte differentiation antigen CD14 precursor, pappalysin-1 preproprotein, phosphatidylinositol-glycan-specific phospholipase D precursor, pigment epithelium-derived factor precursor, plasma kallikrein preproprotein, plasma protease C1 inhibitor precursor, plasminogen isoform 1 precursor, platelet basic protein preproprotein, platelet glycoprotein V precursor, pregnancy zone, protein precursor, pregnancy-specific beta-1-glycoprotein 5, pregnancy-specific beta-1-glycoprotein 5 precursor, pregnancy-specific beta-1-glycoprotein 6, pregnancy-specific beta-1-glycoprotein 6 precursor, pregnancy-specific beta-1-glycoprotein 7, pregnancy-specific beta-1-glycoprotein 8, pregnancy-specific beta-1-glycoprotein 9, pregnancy-specific beta-1-glycoprotein 11, pregnancy-specific beta-1-glycoprotein 2, pregnancy-specific beta-1-glycoprotein 3, pregnancy-specific beta-1-glycoprotein 4, progesterone-induced-blocking factor 1, protein AMBP preproprotein, protein CBFA2T2 isoform MTGR lb, protein FAM98C, protein NLRC3, protein Z-dependent protease inhibitor precursor, prothroinbin preproprotein, putative hydroxypyruvate isomerase isoform 1, ras-like protein family member 10A precursor, ras-related GTP-binding protein A, retinol-binding protein 4 precursor, sex hormone-binding globulin isoform 1 precursor, sex hormone-binding globulin isoform 4 precursor, signal transducer and activator of transcription 2, spectrin beta chain non-erythrocytic 1, stabilin-1 precursor, succinate semialdehyde dehydrogenase mitochondrial, tetranectin precursor, THAP domain-containing protein 6, thyroxine-binding globulin precursor, tripartite motif-containing protein 5, vitamin D-binding protein isoform 1 precursor, vitronectin precursor, zinc finger protein 142, attractin isoform 2 preproprotein, transforming growth factor-beta-induced protein ig-h3 precursor, transthyretin precursor, uncharacterized protein C3orf20, beta-2-microglobulin precursor, bone marrow proteoglycan isoform preproprotein, chorionic gonadotropin beta polypeptide 8 precursor, chorionic somatomammotropin hormone 2 isoform 2 precursor, macrophage colony-stimulating factor 1 receptor precursor, zinc-alpha-2-glycoprotein precursor, PAN-PSG, complement component C6 precursor, EGF-containing fibulin-like extracellular matrix protein 1, and disintegrin and metalloproteinase domain-containing protein 12. Ratios can be obtained by pairing these biomarkers with the steroids described above. Methods of measuring concentrations of these biomarkers in pregnant females are described Saade et al., Am J of Obstetrics & Gynecology, May 2016 633e1-633e24, which is incorporated by reference to the extent it is consistent herewith. Still other suitable biomarkers include sex hormone binding globulin (SHBG), insulin-like growth factor binding protein 4 (IBP4), heat shock protein-70 (Hsp70), heat shock protein-90 (Hsp90).

The method can further include determining a change in nucleic acids of the pregnant female. More particularly, nucleic acids for combinatorial use in the methods of the present disclosure can include nucleic acid primers and/or probes that bind with specific nucleic acid sequences as well as the nucleic acids that are increased or decreased in concentration in pregnant females that are susceptible to preterm delivery. In suitable embodiments, the nucleic acids can include cell free plasma (CFP) RNA such as disclosed in U.S. Publication No. 2015/0376709 to Dong et al. (Sep. 11, 2015), which is incorporated by reference to the extent it is consistent herewith. In other embodiments, the nucleic acids can include cell free fetal DNA (“fetal fraction”). In yet other embodiments, the nucleic acids can include one or more of mRNA, corticotropin releasing hormone (CRH), CRH receptors, calmodulin 1 (CALM1), calmodulin 2 (CALM2), calmodulin 3 (CALM3), placental corticotropin releasing hormone (placental CRH), and (GABA) receptors.

Advantageously, by measuring changes in nucleic acid levels, the efficacy of any treatment given to women with a “positive” score can be assessed. Accordingly, in one embodiment, the methods further include detecting a first concentration of at least one of calmodulin 1 (CALM1), calmodulin 2 (CALM2), calmodulin 3 (CALM3), and placental corticotropin releasing hormone (placental CRH), prior to administering a treatment compounds, and detecting at least a second concentration of at least one of CALM1, CALM2, CALM3, placental corticotropin releasing hormone (placental CRH), after administration of the compound, and wherein an increase in the second concentration of one or more of CALM1, CALM2, CALM3, placental corticotropin releasing hormone (placental CRH), indicates a need to continue administration of the compound to the pregnant female.

The sample can be obtained at gestational times ranging from about 8 weeks to about 41 weeks. In one embodiment, the sample is obtained at a gestational age ranging from about 8 weeks to about 24 weeks, including about 10 weeks to about 24 weeks, including about 10 weeks to about 16 weeks, and including about 10 weeks to about 12 weeks. In another embodiment, the sample is obtained at a gestational age ranging from about 25 weeks to about 35 weeks. In one embodiment, the sample is obtained at less than 34 weeks gestation, including less than 32 weeks. In another embodiment, the sample is obtained at less than 28 weeks gestation. In another embodiment, the sample is obtained at less than 16 weeks.

In one embodiment, the sample is obtained from the pregnant female in the first trimester, generally considered from the date of the last menstrual period to 13 weeks. In one embodiment, the sample is obtained from the pregnant female in the second trimester, generally considered from about the 14^{th} week to about the 27^{th} week. In one embodiment, the sample is obtained from the pregnant female in the third trimester, generally considered from about the 28^{th} week to about the 42^{nd} week.

Suitable samples include a plasma sample, a serum sample, a whole blood sample, a salivary sample and a urine sample. Plasma samples and urine samples are particularly suitable.

The method can further include analyzing at least one pregnancy risk factor. Suitable risk factors include, for example, age, prior pregnancy, history of previous low birth weight or preterm delivery, multiple 2nd trimester spontaneous abortion, prior first trimester induced abortion, preeclampsia, familial and intergenerational factors, history of infertility, nulliparity, placental abnormalities, cervical and uterine anomalies, gestational bleeding, intrauterine growth restriction, in utero diethylstilbestrol exposure, multiple gestations, infant sex, short stature, low pre-pregnancy weight/low body mass index, diabetes, hypertension, hypothyroidism, asthma, education level, tobacco use, and urogenital infections.

Suitable methods for determining concentrations of steroids and biomarkers can be, for example, immunoassays, chromatography, mass spectrometry, amplification, microarray analysis, and combinations thereof. A particularly suitable chromatography-mass spectrometry method includes ultraperformance liquid chromatography-tandem mass spectrometry (UPLC/MS-MS). Particularly suitable immunoassay methods include, for example, enzyme-linked immunosorbent assay (ELISA), Western blot, sandwich immunoassay. Other suitable methods for determining concentrations of steroids and biomarkers include, for example, electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry.

The concentration of 11-deoxycorticosterone is determined using an assay that contacts the sample with an antibody that specifically binds to 11-deoxycorticosterone.

The concentration of isopregnanolone is determined using an assay that contacts the sample with an antibody that specifically binds to isopregnanolone.

The concentration of the steroid in the denominator of the formula is determined using an assay that contacts the sample with an antibody that specifically binds to the steroid, By way of example, when the steroid is 16α-hydroxyprogesterone, the concentration of 16α-hydroxyprogesterone is determined using an assay that contacts the sample with an antibody that specifically binds to 16α-hydroxyprogesterone. By way of another example, when the steroid is 11-deoxycortisol, the concentration of 11-deoxycortisol is determined using an assay that contacts the sample with an antibody that specifically binds to 11-deoxycortisol.

Suitable assays for contacting antibodies that specifically bind to various steroids discussed above include enzyme immunoassay (EIA), enzyme-linked immunosorbent assay (ELISA), and radioimmunoassay (RIA).

The method can further include analyzing at least one pregnancy risk factor. Suitable risk factors include, for example, age, race, medication exposure (e.g., administration or previous administration to (e.g., 17 hydroxyprogesterone, progesterone), prior pregnancy, history of previous low birth weight or preterm delivery, multiple 2nd trimester spontaneous abortion, prior first trimester induced abortion, preeclampsia, familial and intergenerational factors, history of infertility, nulliparity, placental abnormalities, cervical and uterine anomalies, gestational bleeding, intrauterine growth restriction, in utero diethylstilbestrol exposure, multiple gestations, infant sex, short stature, low pre-pregnancy weight/low body mass index, diabetes, hypertension, hypothyroidism, asthma, education level, tobacco use, and urogenital infections.

The method can further include determining a concentration of at least one additional biomarker as described herein.

In other embodiments, the present disclosure is directed to treating a pregnant female upon analyzing the hormone profile of the female. Generally, the disclosure provides for a method for treating a pregnant female that is identified as being susceptible to spontaneous preterm delivery based on the calculated score of the formula used in the methods for analyzing the hormone profile. The pregnant female can generally be treated with an agent that can antagonize the glucocorticoid pathway. Exemplary compounds include progesterone, 17α-hydroxyprogesterone caproate (HPC), monohydroxylated HPC (HPC-OH), atosiban, nifedipine, terbutaline, eplerenone, fluoxetine, norfluoxetine, other mineralocorticoid specific antagonists, modulators of cytochrome P450 function (e.g., CYP2D6, CYP2C9, CYP3A4), and derivatives thereof.

By way of example, the methods allow for the administration of a treatment if the score calculated using the above-described formula is less than 1.5, including less than 1.1. More particularly, in one embodiment, when the formula is

$$Score = \frac{[ 11 - deoxycorticosterone ]}{{( {log}_{10} [ 16 α - hydroxyprogesterone ] )}^{l o {g}_{10} [ isopre g n anolone ]}} ,$$

the methods allow for administration of a treatment if the calculated score is less than 1.5, including less than 1.2, and including less than 1.0. In another embodiment, when the formula is

$$Score = \frac{[ 11 - deoxycorticosterone ]}{{( {log}_{10} [ 11 - deoxycortisol ] )}^{l o {g}_{10} [ isopre g n anolone ]}} ,$$

the methods allow for administration of a treatment if the calculated score is less than 1.0, including less than 0.8, and including less than 0.6.

In particular, if the calculated score of the formula used in the methods of the present disclosure is less than 1.1, then the methods provide for administering an effective amount of the compound (i.e., 17α-hydroxyprogesterone caproate (HPC), HPC-OH, atosiban, nifedipine, terbutaline, eplerenone, fluoxetine, norfluoxetine, other mineralocorticoid specific antagonists, modulators of cytochrome P450 function (e.g., CYP2D6, CYP2C9, CYP3A4), and derivatives thereof.

In an embodiment, the treatment methods described herein provide for a method of treating a pregnant female with one or more compounds selected progesterone, 17α-hydroxyprogesterone caproate (HPC), monohydroxylated HPC (HPC-OH), atosiban, nifedipine, terbutaline, eplerenone, fluoxetine, norfluoxetine, other mineralocorticoid specific antagonists, modulators of cytochrome P450 function (e.g., CYP2D6, CYP2C9, CYP3A4), and derivatives thereof. As used herein, “treatment” or “treating” or “treat” refers to prophylactic treatment and/or treatment that allows for reduction or completely halting of the symptoms of spontaneous preterm delivery (e.g., uterine contractility, cervical shortening or change).

In an embodiment, the steroids for use in the treatment methods include those described above. Particularly suitable steroids include, for example, deoxycorticosterone, corticorterone, 18-hydroxycortiosterone, aldosterone, deoxycortisol, cortisol, and combinations thereof. Suitable steroids can also be selected from progesterone, 16α-hydroxyprogesterone, 6β-hydroxyprogesterone, 6α-hydroxyprogesterone, 17-hydroxyprogesterone, 11-deoxycortisol, cortisol, 11-deoxycorticosterone, 17-deoxycortisol, androstenedione, testosterone, estradiol, 20α-dihydroprogesterone, 17α,20α-dihydroxyprogesterone, isopregnanolone, and combinations thereof. By way of example, in some embodiments, the first steroid is deoxycorticosterone (DOC) and the second steroid is 16α-hydroxyprogesterone (16αOHP). In other embodiments, the first steroid is deoxycorticosterone (DOC) and the second steroid is 11-deoxycortisol.

The neurosteriod is selected from isopregnanolone, epipregnanolone, pregnenolone sulfate, pregnanolone, allopregnanolone, tetrahydrodeoxycorticosterone, and combinations thereof.

The treatment methods can further include determining a change in a concentration of at least one additional biomarker selected from insulin-like growth factor binding protein 4, sex-hormone binding globulin (SHBG), lipopolysaccharide-binding protein (LBP), lipopolysaccharide-binding protein (LBP) precursor, prothrombin (THRB), complement component C5 (C5 or CO5), plasminogen (PLMN), complement component C8 gamma chain (C8G or CO8G), Complement factor B, Ectonucleotide pyrophosphatase/phosphodiesterase family member 2, Gelsolin, N-acetylmuramoyl-L-alanine amidase, N-acetylmuramoyl-L-alanine amidase precursor, Hyaluronan-binding protein 2, BPI fold-containing family B member 1, complement component C8 alpha chain, apolipoprotein A-II, Ectonucleotide pyrophosphatase/phosphodiesterase family member 2, profiling-1, pro-neuropeptide Y, complement component C8 beta chain, coagulation factor XIIII B chain, N-acetylmuramoyl-L-alanine amidase, inter-alpha-trypsin inhibitor heavy chain H4, inter-alpha-trypsin inhibitor heavy chain H3 preproprotein, leucyl-cystinyl aminopeptidase, alpha-2-HS-glycoprotein, 5′-AMP-activated protein kinase subunit gamma-3, afamin precursor, alpha-1l-antichymotrypsin precursor, alpha-1B-glycoprotein precursor, alpha-2-antiplasmin isoform a precursor, alpha-2-HS-glycoprotein preproprotein, alpha-2-HS-macroglobulin precursor, angiotensinogen preproprotein, antithrombin-III precursor, apolipoprotein A-II preproprotein, apolipoprotein A-IV precursor, apolipoprotein B-100 precursor, apolipoprotein C-I precursor, apolipoprotein C-II precursor, apolipoprotein C-III precursor, apolipoprotein E precursor, ATP-binding cassette sub-family D member 4, ATP-binding cassette sub-family F member 3, beta-2-glycoprotein 1 precursor, beta-Ala-His dipeptidase precursor, biotinidase precursor, carboxypeptidase B2 preproprotein, carboxypeptidase N catalytic chain precursor, carboxypeptidase N subunit 2 precursor, catalase, ceruloplasmin precursor, cholinesterase precursor, clusterin preproprotein, coagulation factor IX preproprotein, coagulation factor VII isoform a, coagulation factor VII isoform a preproprotein, coagulation factor X preproprotein, coagulation factor XIII B chain, coiled-coil domain-containing protein 13, complement C1q subcomponent subunit A precursor, complement C1q subcomponent subunit B precursor, complement C1q subcomponent subunit C precursor, complement C1r subcomponent precursor, complement C1s subcomponent precursor, complement C2 isoform 3, complement C3 precursor, complement C4-A isoform 1, complement C5 preproprotein, component C6 precursor, component C7 precursor, component C8 alpha chain precursor, complement component C9 precursor, complement factor B preproprotein, complement factor H isoform a precursor, complement factor H isoform b precursor, complement factor H H-related protein 1 precursor, complement factor I preproprotein, conserved oligomeric Golgi complex subunit 6 isoform, corticosteroid-binding globulin precursor, C-reactive protein precursor, dopamine beta-hydroxylase precursor, double-stranded RNA-specific editase B2, dual oxidase 2 precursor, FERM domain-containing protein 8, fetuin-B precursor, ficolin-3 isoform 1 precursor, gastric intrinsic factor precursor, gelsolin isoform d, glutathione peroxidase 3 precursor, hemopexin precursor, heparin cofactor 2 precursor, hepatocyte cell adhesion molecule precursor, hepatocyte growth factor activator preproprotein, histidine-rich glycoprotein precursor, hyaluronan-binding protein 2 isoform 1 preproprotein, inactive caspase-12 insulin-degrading enzyme isoform 1, insulin-like growth factor-binding protein complex acid labile subunit isoform 2 precursor, inter-alpha-trypsin inhibitor heavy chain H1 isoform a precursor, inter-alpha-trypsin inhibitor heavy chain H2 precursor, inter-alpha-trypsin inhibitor heavy chain H4 isoform 1 precursor, kallistatin precursor, kininogen-1 isoform 2 precursor, leucine-rich alpha-2-glycoprotein precursor, lumican precursor, m7GpppX diphosphatase, matrix metalloproteinase-19 isoform 1 preproprotein, MBT domain-containing protein 1, monocyte differentiation antigen CD14 precursor, pappalysin-1 preproprotein, phosphatidylinositol-glycan-specific phospholipase D precursor, pigment epithelium-derived factor precursor, plasma kallikrein preproprotein, plasma protease C1 inhibitor precursor, plasminogen isoform 1 precursor, platelet basic protein preproprotein, platelet glycoprotein V precursor, pregnancy zone protein precursor, pregnancy-specific beta-1-glycoprotein 5, pregnancy-specific beta-1-glycoprotein 5 precursor, pregnancy-specific beta-1-glycoprotein 6, pregnancy-specific beta-1-glycoprotein 6 precursor, pregnancy-specific beta-1-glycoprotein 7, pregnancy-specific beta-1-glycoprotein 8, pregnancy-specific beta-1-glycoprotein 9, pregnancy-specific beta-1-glycoprotein 11, pregnancy-specific beta-1-glycoprotein 2, pregnancy-specific beta-1-glycoprotein 3, pregnancy-specific beta-1-glycoprotein 4, progesterone-induced-blocking factor 1, protein AMBP preproprotein, protein CBFA2T2 isoform MTGR1b, protein FAM98C, protein NLRC3, protein Z-dependent protease inhibitor precursor, prothrombin preproprotein, putative hydroxypyruvate isomerase isoform 1, ras-like protein family member 10A precursor, ras-related GTP-binding protein A, retinol-binding protein 4 precursor, sex hormone-binding globulin isoform 1 precursor, sex hormone-binding globulin isoform 4 precursor, signal transducer and activator of transcription 2, spectrin beta chain non-erythrocytic 1, stabilin-1 precursor, succinate semialdehyde dehydrogenase mitochondrial, tetranectin precursor, THAP domain-containing protein 6, thyroxine-binding globulin precursor, tripartite motif-containing protein 5, vitamin D-binding protein isoform 1 precursor, vitronectin precursor, zinc finger protein 142, attractin isoform 2 preproprotein, transforming growth factor-beta-induced protein ig-h3 precursor, transthyretin precursor, uncharacterized protein C3orf20, beta-2-microglobulin precursor, bone marrow proteoglycan isoform 1 preproprotein, chorionic gonadotropin beta polypeptide 8 precursor, chorionic somatomammotropin hormone 2 isoform 2 precursor, macrophage colony-stimulating factor 1 receptor precursor, zinc-alpha-2-glycoprotein precursor, PAN-PSG, complement component C6 precursor, EGF-containing fibulin-like extracellular matrix protein 1, and disintegrin and metalloproteinase domain-containing protein 12. Ratios can be obtained by pairing these biomarkers with the steroids described above. Methods of measuring concentrations of these biomarkers in pregnant females are described Saade et al., Am J of Obstetrics & Gynecology, May 2016 633e1-633e24, which is incorporated by reference to the extent it is consistent herewith. Other suitable biomarkers include sex hormone binding globulin (SHBG), insulin-like growth factor binding protein 4 (IBP4), heat shock protein-70 (Hsp70), and heat shock protein-90 (Hsp90).

The method can further include determining a change in nucleic acids of the pregnant female. More particularly, nucleic acids for combinatorial use in the methods of the present disclosure can include nucleic acid primers and/or probes that bind with specific nucleic acid sequences as well as the nucleic acids that are increased or decreased in concentration in pregnant females that are susceptible to preterm delivery. In suitable embodiments, the nucleic acids can include cell free plasma (CFP) RNA such as disclosed in U.S. Publication No. 2015/0376709 to Dong et al. (Sep. 11, 2015). In other embodiments, the nucleic acids can include cell free fetal DNA (“fetal fraction”). In yet other embodiments, the nucleic acids can include corticotropin releasing hormone (CRH), CRH receptors, calmodulin 1 (CALM1), calmodulin 2 (CALM2), calmodulin 3 (CALM3), placental corticotropin releasing hormone (placental CRH), and (GABA) receptors.

The sample can be obtained at gestational times ranging from about 8 weeks to about 41 weeks. In one embodiment, the sample is obtained at a gestational age ranging from about 8 weeks to about 24 weeks. In one embodiment, the sample is obtained at a gestational age ranging from about 8 weeks to about 24 weeks, including about 10 weeks to about 24 weeks, including about 10 weeks to about 16 weeks, and including above 10 weeks to about 12 weeks. In another embodiment, the sample is obtained at a gestational age ranging from about 25 weeks to about 35 weeks. In one embodiment, the sample is obtained at less than 34 weeks gestation, including less than 32 weeks. In another embodiment, the sample is obtained at less than 28 weeks gestation. In another embodiment, the sample is obtained at less than 16 weeks.

In one embodiment, the sample is obtained from the pregnant female in the first trimester, generally considered from the date of the last menstrual period to 13 weeks. In one embodiment, the sample is obtained from the pregnant female in the second trimester, generally considered from about the 14th week to about the 27th week. In one embodiment, the sample is obtained from the pregnant female in the third trimester, generally considered from about the 28th week to about the 42nd week.

Suitable samples include a plasma sample, a serum sample, a whole blood sample, salivary sample, and a urine sample. Plasma samples and urine samples are particularly suitable.

The methods can further include analyzing at least one pregnancy risk factor. Suitable risk factors include, for example, age, prior pregnancy, history of previous low birth weight or preterm delivery, multiple 2nd trimester spontaneous abortion, prior first trimester induced abortion, preeclampsia, familial and intergenerational factors, history of infertility, environmental factors, nulliparity, placental abnormalities, cervical and uterine anomalies, gestational bleeding, intrauterine growth restriction, in utero diethylstilbestrol exposure, multiple gestations, infant sex, short stature, low prepregnancy weight/low body mass index, diabetes, hypertension, hypothyroidism, asthma, education level, tobacco use, and urogenital infections.

Suitable methods for determining concentrations of steroids and biomarkers can be, for example, immunoassays, chromatography, mass spectrometry, amplification, microarray analysis, and combinations thereof. A particularly suitable chromatography-mass spectrometry method includes ultraperformance liquid chromatography-tandem mass spectrometry (UPLC/MS-MS). Particularly suitable immunoassay methods include, for example, enzyme-linked immunosorbent assay (ELISA), Western blot, sandwich immunoassay. Other suitable methods for determining concentrations of steroids and biomarkers include, for example, electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry.

The treatment methods include administering an effective amount of one or more of progesterone, 17α-hydroxyprogesterone caproate (HPC), monohydroxylated HPC (HPC-OH), atosiban, nifedipine, terbutaline, eplerenone, fluoxetine, norfluoxetine, other mineralocorticoid specific antagonists, modulators of cytochrome P450 function (e.g., CYP2D6, CYP2C9, CYP3A4), and derivatives thereof to the female patient when the calculated score is less than 1.5, suitably, less than 1.1.

In particularly suitable embodiments, one or more of 17α-hydroxyprogesterone caproate (HPC), monohydroxylated HPC (HPC-OH), atosiban, nifedipine, terbutaline, eplerenone, fluoxetine and norfluoxetine, other mineralocorticoid specific antagonists, modulators of cytochrome P450 function (e.g., CYP2D6, CYP2C9, CYP3A4), and derivatives thereof is administered to the female patient after 10 weeks of gestation, suitably, after 12 weeks of gestation and even more suitably, at or after 16 weeks gestation.

Suitable dosages of 17α-hydroxyprogesterone caproate (HPC), monohydroxylated HPC (HPC-OH), atosiban, nifedipine, terbutaline, eplerenone, fluoxetine and norfluoxetine, other mineralocorticoid specific antagonists, modulators of cytochrome P450 function (e.g., CYP2D6, CYP2C9, CYP3A4), and derivatives thereof thereof may be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

Suitable amounts of one or more of 17α-hydroxyprogesterone caproate (HPC), monohydroxylated HPC (HPC-OH), atosiban, nifedipine, terbutaline, eplerenone, fluoxetine and norfluoxetine, other mineralocorticoid specific antagonists, modulators of cytochrome P450 function (e.g., CYP2D6, CYP2C9, CYP3A4), and derivatives thereof for use in the dosage forms of the present disclosure will depend upon many factors including, for example, age and weight of an pregnant female, specific compound(s) to be used, nature of a composition, whether the composition is intended for direct administration or is a concentrate, and combinations thereof.

The methods can further include analyzing administering or modifying a life factor selected from exercise regimen, dietary regimen, sleep patterns, and smoking cessation.

Suitable female subjects include, but are not limited to, a human female, a livestock female animal, a companion female animal, a lab female animal, and a zoological female animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non- limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In another embodiment, the animal is a laboratory animal. Non-limiting examples of a laboratory animal may include rodents, canines, felines, and non-human primates. In certain embodiments, the animal is a rodent. In a further embodiment, the subject is human.

In the above formulas, the steroid for use in the denominator of the formula includes one or more of 16α-hydroxyprogesterone, corticosterone, 18-hydroxycortiosterone, aldosterone, 11-deoxycortisol, estradiol, testosterone, and 17-hydroxyprogesterone, cortisol. In particular, the steroid can be one or more of 16α-hydroxyprogesterone, 11-deoxycortisol, estradiol, testosterone, and 17-hydroxyprogesterone.

The neurosteriod is selected from isopregnanolone, epipregnanolone, pregnenolone sulfate, pregnanolone, allopregnanolone, tetrahydrodeoxycorticosterone, and combinations thereof.

Suitable samples include a plasma sample, a serum sample, a whole blood sample, a salivary sample and a urine sample. Plasma samples and urine samples are particularly suitable.

Suitable methods for determining concentrations of steroids and biomarkers can be, for example, immunoassays, chromatography, mass spectrometry, amplification, microarray analysis, and combinations thereof. A particularly suitable chromatography-mass spectrometry method includes ultraperformance liquid chromatography-tandem mass spectrometry (UPLC/MS-MS). Particularly suitable immunoassay methods include, for example, enzyme-linked immunosorbent assay (ELISA), Western blot, sandwich immunoassay. Other suitable methods for determining concentrations of steroids and biomarkers include, for example, electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry.

The concentration of 11-deoxycorticosterone is determined using an assay that contacts the sample with an antibody that specifically binds to 11-deoxycorticosterone.

The concentration of isopregnanolone is determined using an assay that contacts the sample with an antibody that specifically binds to isopregnanolone.

The concentration of the steroid in the denominator of the formula is determined using an assay that contacts the sample with an antibody that specifically binds to the steroid, By way of example, when the steroid is 16α-hydroxyprogesterone, the concentration of 16α-hydroxyprogesterone is determined using an assay that contacts the sample with an antibody that specifically binds to 16α-hydroxyprogesterone. By way of another example, when the steroid is 11-deoxycortisol, the concentration of 11-deoxycortisol is determined using an assay that contacts the sample with an antibody that specifically binds to 11-deoxycortisol.

Suitable assays for contacting antibodies that specifically bind to various steroids discussed above include enzyme immunoassay (EIA), enzyme-linked immunosorbent assay (ELISA), and radioimmunoassay (RIA).

### EXAMPLES

### Example 1

In this Example, the association of selected endogenous steroids to risk of spontaneous (very spontaneous; less than 32 weeks) preterm delivery was analyzed. Particularly, the association of endogenous steroids, including neurosteroids, estrogens, and androgens, were analyzed.

The steroid and neurosteroid molecules targeted for quantification included: testosterone, estradiol, and isopregnanolone. These quantified molecules were used to modify a previously established equation for risk stratification of women suspected of being susceptible to spontaneous very preterm delivery (sPTD). Prospectively collected plasma specimens from 68 women who delivered preterm (<37 weeks) were obtained for the analysis. Plasma samples were divided into 2 epochs for analysis: Epoch 1 (late first trimester/early second trimester) and Epoch 2 (early third trimester).

A targeted metabolomics approach was used to quantify endogenous progestogen, glucocorticoid, and mineralocorticoid steroids using ultraperformance liquid chromatography-tandem mass spectrometry (UPLC/MS-MS) analysis. The frozen human plasma samples were processed using an assay validated on the mass spectroscopy platform. Briefly, 1-2 mL aliquots of plasma were adjusted to pH 7.0 and subject to solid phase extraction (SPE) with methanol.

The methanol fraction was subjected to ultraperformance liquid chromatography-tandem mass spectrometry (UPLC/MS-MS) analysis. Analytical separations on the UPLC system were conducted with C18 or phenyl columns (1×100 mm). The elutions from the UPLC column were introduced to the mass spectrometer. All MS experiments were performed by using electrospray ionization (ESI) in positive ion (PI) and negative ion (NI) mode. Using pure reference standards, the multiple reaction monitoring method (MRM) for UPLC/MS-MS operation was generated. Pure standards were used to optimize the UPLC-MS/MS conditions prior to analysis and make calibration curves. Elutions from UPLC column were analyzed in the MRM mode, and resulting data was processed using standard analytical software.

Data from quantification of the endogenous steroids was used to identify molecules associated with sPTD (<32 weeks). Statistical analysis was performed using SPSS Statistics Version 26 software (IBM, Armonk, N.Y.). Receiver operating characteristic (ROC) curves were used to identify steroid hormone patterns capable of distinguishing patients who would subsequently have sPTD (<32 weeks). Area under the ROC curve (AUROC) was used to compare the predictive ability of individual and combinations of steroid hormones in Epochs 1 and 2. Patient demographic and clinical outcome data was analyzed using standard statistical approaches.

### Results

Of the 68 subjects in the preterm (<37 weeks) delivery cohort, 28 subjects (41%) delivered spontaneously in the very or extreme preterm period (vePTD, <32 weeks), while the remaining 40 (59%) delivered in the moderate to late preterm period (modPTD, 33-37 weeks). Plasma samples were obtained from subjects at a mean gestational age of 15.9±3.7 weeks in the vePTD group and 15.6±4.4 weeks in the modPTD group in Epoch 1 (p=0.775).

In Epoch 2, samples were obtained at a mean gestational age of 28.5±2.0 weeks in the vePTD group and 30.1±3.5 weeks in the modPTD group (p=0.065).

Patient demographics were similar between the vePTD and modPTD groups (see Table 1). There were no differences in maternal age, race, BMI, or history of prior PTD between groups. Maternal co-morbidities were more frequently observed in the modPTD group compared to the vePTD group (p=0.005). Antepartum complications were similar between groups (Table 1).

TABLE 1

| | Gestational Age at Delivery | | |
|---|---|---|---|
| | (weeks) | | P |
| Parameter | <32 | 33-37 | value |
| N (%) | 28 (30%) | 40 (43%) | |
| Age (years) | 25.5 | 27.6 | 0.21 |
| BMI | 32 | 32 | 0.97 |
| History of PTD, | 36% | 43% | 0.81 |
| % | | | |
| Race (%) | | | 0.37¹ |
| Caucasian | 13 (46%) | 23 (57%) | |
| African | 14 (50%) | 17 (43%) | |
| American | | | |
| Not reported | 1 (4%) | 0 | |
| Composite | 0.7 | 1.5 | 0.005 |
| Maternal² | | | |
| Composite | 0.9 | 1.1 | 0.57 |
| Antepartum³ | | | |

¹Analysis by Chi-square; all others by T-test.
²Average number of pre-specified maternal co-morbidities (see methods).
³Average number of pre-specified antepartum complications (see methods).

Mathematical transformation of endogenous steroid concentrations appeared to improve the ability of the algorithm to predict preterm birth <32 weeks. Surprisingly, use of the logarithm function in conjunction with raising the endogenous steroid concentration to the power defined by the logio of isopregnanolone optimized the predictive characteristics of the algorithm. Use of the logarithm function allowed for reduction in dispersion and skew of the concentrations. By assessing a variety of transformations, it became evident that the following equation optimized prediction of spontaneous preterm delivery at <32 weeks from samples in the first or second trimester:

$$Risk Score = \frac{[ 11 - deoxycorticosterone ]}{{( {log}_{10} [ 16 α - Hydroxyprogesterone ] )}^{l o {g}_{10} [ Isopregnanolone ]}} \qquad Eq . 1$$

Within the preterm delivery cohort (n=68), Equation 1 provided a Risk Score that yielded a ROC curve with an AUC of 0.830 (p=0.002). A threshold score of 1.09 provided 100% sensitivity, 54% specificity for the prediction of spontaneous vePTD. Equation 1 was associated with a PPV 35% and NPV 100% for spontaneous vePTD. Subjects with a Risk Score below the threshold value (1.09) delivered at a mean gestational age of 30±4.8 weeks vs. 33.3±2.7 weeks for subjects with scores above 1.09 (p=0.006). Equation 1 retained its ability to identify any vePTD (spontaneous+iatrogenic) within the cohort as well (p=0.002).

The inclusion of isopregnanolone into the algorithm implicates a neurosteroid with a clearly defined role in maternal stress response. Isopregnanolone is an antagonist to allopregnanolone, which acts as a natural anxiolytic or stress-reducer hormone. Thematically, mineralocorticoids, 16a-OHP, and isopregnanolone point to the importance of stress-related pathways in the prediction of PTB<32 weeks.

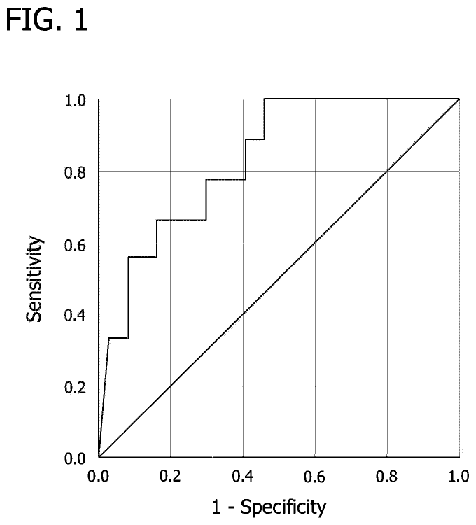

FIG. 1 is a ROC graph depicting various ratios of 11-deoxycorticosterone (cortexone) and/or 16α-Hydroxyprogesterone (16α-OHP) and isopregnanolone and spontaneous preterm birth (sPTB) at less than 32 weeks in Epoch 1. AUC 0.830, 95% CI 0.697-0.963 (p=0.002),100% sensitivity, 54% specificity (threshold≤1.09).

1. A method for analyzing a hormone panel in a pregnant female, the method comprising:
obtaining a sample from the pregnant female;
detecting a concentration of 11-deoxycorticosterone in the sample;
detecting a concentration of a neurosteroid in the sample;
detecting a concentration of a steroid in the sample; and
calculating a score for the hormone profile using the formula of:
$$Score = \frac{[ 11 - deoxycorticosterone ]}{{( \frac{{\sum }_{i = 1}^{j} {{log}_{10} [ steroid ]}_{i}}{j} )}^{{\prod }_{i = 1}^{p} {{log}_{10} [ neurosteroid ]}_{i}}}$$
wherein j is the number of steroid molecules measured; p represents the number of neurosteroid molecules measured; and i represents individual steroid or neurosteroid molecules up to a total of j or p, respectively.

2. The method of claim 1, wherein the steroid is selected from the group consisting of 16α-hydroxyprogesterone, corticosterone, 18-hydroxycortiosterone, aldosterone, 11-deoxycortisol, estradiol, testosterone, and 17-hydroxyprogesterone, cortisol, and combinations thereof.

3. The method of claim 1, wherein the neurosteroid is selected from the group consisting of isopregnanolone, epipregnanolone, pregnenolone sulfate, pregnanolone, allopregnanolone, tetrahydrodeoxycorticosterone, and combinations thereof.

4. The method of claim 1, further comprising determining a concentration of a biomarker selected from the group consisting of sex hormone binding globulin (SHBG), insulin-like growth factor binding protein 4 (IBP4), heat shock protein-70 (Hsp70), heat shock protein-90 (Hsp90), and combinations thereof.

5. The method of claim 1, further comprising determining a change in a concentration of a nucleic acid.

6. The method of claim 5, where the nucleic acid is at least one of cell free plasma (CFP) RNA, mRNA, cell free fetal DNA, corticotropin releasing hormone (CRH), CRH receptors, calmodulin 1 (CALM1), calmodulin 2 (CALM2), calmodulin 3 (CALM3), and gamma-aminobutyric acid (GABA) receptors.

7. The method of claim 1, wherein the pregnant female is in the first trimester and calculating the score comprises using the formula:
$$Score = \frac{[ 11 - deoxycorticosterone ]}{{( {log}_{10} [ 16 α - hydroxyprogesterone ] )}^{l o {g}_{10} [ isopre g n anolone ]}} .$$

8. The method of claim 7, wherein the concentration of 11-deoxycorticosterone is determined using an assay that contacts the sample with an antibody, wherein the antibody specifically binds to 11-deoxycorticosterone.

9. The method of claim 7, wherein the concentration of 16α-hydroxyprogesterone is determined using an assay that contacts the sample with an antibody that specifically binds to 16α-hydroxyprogesterone.

10. The method of claim 7, wherein the concentration of isopregnanolone is determined using an assay that contacts the sample with an antibody that specifically binds to isopregnanolone.

11. The method of claim 1, wherein the pregnant female is in the second trimester and calculating the score comprises using the formula:
$$Score = \frac{[ 11 - deoxycorticosterone ]}{{( {log}_{10} [ 16 α - hydroxyprogesterone ] )}^{l o {g}_{10} [ isopre g n anolone ]}} .$$

12. The method of claim 11, wherein the concentration of 11-deoxycorticosterone is determined using an assay that contacts the sample with an antibody, wherein the antibody specifically binds to 11-deoxycorticosterone.

13. The method of claim 11, wherein the concentration of 16α-hydroxyprogesterone is determined using an assay that contacts the sample with an antibody that specifically binds to 16α-hydroxyprogesterone.

14. The method of claim 11, wherein the concentration of isopregnanolone is determined using an assay that contacts the sample with an antibody that specifically binds to isopregnanolone.

15. The method of claim 1, wherein the pregnant female is in the second trimester and calculating the score comprises using the formula:
$$Score = \frac{[ 11 - deoxycorticosterone ]}{{( {log}_{10} [ estradiol ] )}^{l o {g}_{10} [ isopre g n anolone ]}} .$$

16. The method of claim 1, wherein the sample is obtained at less than 32 weeks.

17. (canceled)

18. (canceled)

19. The method of claim 1, wherein the sample is selected from a plasma sample, a serum sample, a whole blood sample, a salivary sample, and a urine sample.

20. The method of claim 1 further comprising treating the pregnant female, the method comprising: administering an effective amount of a compound selected from the group consisting of 17α-hydroxyprogesterone caproate (HPC), monohydroxylated HPC (HPC-OH), atosiban, nifedipine, terbutaline, eplerenone, allopregnanolone, fluoxetine, norfluoxetine, CYP2D6, CYP2C9, CYP3A4 and combinations thereof if the score is less than 1.5.

21. The method of claim 20, further comprising detecting a first concentration of at least one of calmodulin 1 (CALM1), calmodulin 2 (CALM2), calmodulin 3 (CALM3), and placental corticotropin releasing hormone (placental CRH), prior to administering the compound, and detecting at least a second concentration of at least one of CALM1, CALM2, CALM3, placental corticotropin releasing hormone (placental CRH), after administration of the compound, and wherein an increase in the second concentration of one or more of CALM1, CALM2, CALM3, placental corticotropin releasing hormone (placental CRH), indicates a need to continue administration of the compound to the pregnant female.

22. The method of claim 20, further comprising analyzing at least one risk factor selected from the group consisting of: age, race, medication exposure, prior pregnancy, history of previous low birth weight or preterm delivery, multiple 2nd trimester spontaneous abortion, prior first trimester induced abortion, preeclampsia, familial and intergenerational factors, history of infertility, nulliparity, placental abnormalities, cervical and uterine anomalies, gestational bleeding, intrauterine growth restriction, in utero diethylstilbestrol exposure, multiple gestations, infant sex, short stature, low pre-pregnancy weight/low body mass index, diabetes, hypertension, hypothyroidism, asthma, education level, tobacco use, urogenital infections, activity levels, sleep quality, and combinations thereof.

23. (canceled)
